(19) United States
(12) Patent Application Publication
(10) Pub. No.: US 2026/0191424 A1
(43) Pub. Date: Jul. 9, 2026

(54) WEARABLE ELECTRONIC DEVICE AND METHOD CAPABLE OF INFORMING DIFFERENT USERS OF APPROPRIATELY WEARING WEARABLE ELECTRONIC DEVICE WITH DIFFERENT LEVELS OF TIGHTNESS TO DETECT ACCURATE PHYSIOLOGICAL FEATURES

(71) Applicant: PixArt Imaging Inc., Hsin-Chu City (TW)

(72) Inventors: Chun-Chih Chen, Hsin-Chu City (TW); Yung-Chang Lin, Hsin-Chu City (TW); Jian-Cheng Liao, Hsin-Chu City (TW)

(73) Assignee: PixArt Imaging Inc., Hsin-Chu City (TW)

(21) Appl. No.: 19/014,265

(22) Filed: Jan. 9, 2025

(51) Int. Cl. A61B 5/024; A61B 5/00

(52) U.S. Cl. CPC A61B 5/02427; A61B 5/0026; A61B 5/0059; A61B 5/681

(57) ABSTRACT

A method of a wearable electronic device includes: providing at least one photoplethysmogram (PPG) sensor to sense a physiological feature of a user by measuring at least one PPG signal by illuminating user's skin and measuring changes in light absorption based on light reflected from the user's skin when the wearable electronic device is worn by the user; using at least one pressure sensor, located adjacent to the at least one PPG sensor, to measure at least one contact force between the user's skin and the wearable electronic device when the wearable electronic device is worn by the user; determining at least one reliability level corresponding to the at least one PPG signal according to the at least one measured contact force; and, determining whether to output information of the at least one PPG signal for the user according to the at least one determined reliability level.

### BACKGROUND OF THE INVENTION

### 1. Field of the Invention

The invention relates to a scheme of a wearable electronic device, and more particularly to the wearable electronic device and a corresponding method.

### 2. Description of the Prior Art

Generally speaking, the conventional wearable device products using gravity, optical, or capacitive sensors often fail to measure the contact force between the user's skin and the device, and excessively light or heavy contact force may inevitably reduce the quality of raw PPG (Photoplethysmography) data, thereby affecting the accuracy of health monitoring functions. A raw PPG signal is an optically obtained plethysmogram signal that can be used to detect blood volume changes in the microvascular bed of tissue, and it is often obtained by the conventional wearable device products by illuminating the skin and measuring changes in light absorption.

### SUMMARY OF THE INVENTION

Therefore one of the objectives of the invention is to provide a wearable electronic device and a corresponding method, to solve the above-mentioned problems.

According to embodiments of the invention, a wearable electronic device is disclosed. The wearable electronic device comprises at least one photoplethysmogram (PPG) sensor, at least one pressure sensor, an integration circuit, and a microcontroller. The at least one PPG sensor is used for sensing a physiological feature of a user by measuring at least one PPG signal by illuminating the user's skin and measuring changes in light absorption based on at least one light reflected from the user's skin when the wearable electronic device is worn by the user. The at least one pressure sensor, located adjacent to the at least one PPG sensor, is used for measuring at least one contact force between the user's skin and the wearable electronic device when the wearable electronic device is worn by the user. The integration circuit, coupled to the at least one PPG sensor and the at least one pressure sensor, is used for determining at least one reliability level corresponding to the at least one PPG signal according to the at least one measured contact force. The microcontroller, coupled to the integration circuit, is used for determining whether to output information of the at least one PPG signal for the user according to the at least one determined reliability level.

According to the embodiments, a method of a wearable electronic device is disclosed. The method comprises: providing at least one photoplethysmogram (PPG) sensor to sense a physiological feature of a user by measuring at least one PPG signal by illuminating the user's skin and measuring changes in light absorption based on at least one light reflected from the user's skin when the wearable electronic device is worn by the user; using at least one pressure sensor, located adjacent to the at least one PPG sensor, to measure at least one contact force between the user's skin and the wearable electronic device when the wearable electronic device is worn by the user; determining at least one reliability level corresponding to the at least one PPG signal according to the at least one measured contact force; and, determining whether to output information of the at least one PPG signal for the user according to the at least one determined reliability level.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a block diagram of a wearable electronic device according to an embodiment of the invention.

FIG. 2 is a diagram showing an example of the wearable electronic device being implemented as a smart watch device according to an embodiment of the invention.

FIG. 3 is a diagram showing the comparison between the waveform of measured PPG signal and normal waveform of a corresponding reference PPG signal for the different examples of the moderate contact force and large contact force.

FIG. 4 is a diagram showing an example of the relation between the different oxygen saturation levels and the difference contact force values according to an embodiment of the invention.

FIG. 5 is a block diagram of a wearable electronic device according to another embodiment of the invention.

### DETAILED DESCRIPTION

The invention aims at providing a wearable electronic device and a corresponding method capable of informing different users of appropriately wearing the wearable electronic device with different levels of tightness according to the measured pressures caused by the different users so as to respectively obtain accurate and precise results of physiological features of the different users to improve the accuracy of the detection of the physiological features.

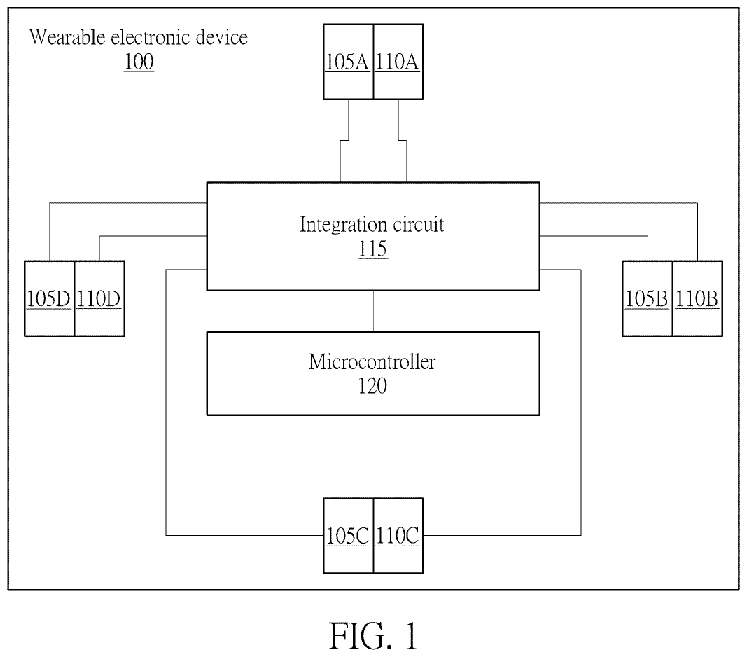

FIG. 1 is a block diagram of a wearable electronic device 100 according to an embodiment of the invention. The wearable electronic device 100 for example comprises multiple photoplethysmogram (PPG) sensors such as four PPG sensors 105A-105D (but not limited), multiple pressure sensors such as four pressure sensors 110A-110D (but not limited), an integration circuit 115, and a microcontroller 120. The number of PPG sensors (or pressure sensors) can be different in different examples and is not intended to be a limitation of the invention. A raw PPG signal is an optically obtained plethysmogram signal that can be used to detect blood volume changes in the microvascular bed of tissue, and it can be generated or obtained by the wearable electronic device 100 by illuminating the user's skin and measuring changes in light absorption.

Each of the PPG sensors 105A-105D is used for sensing a physiological feature (or physiological characteristics, or biological characteristics) of a user by measuring and generating a PPG signal (i.e. a raw PPG signal) by illuminating the user's skin and measuring changes in light absorption based on a light reflected from the user's skin when the wearable electronic device 100 is worn by the user. The light may be emitted from light emission circuits (not shown in FIG. 1) of the PPG sensors 105A-105D into the different points/positions of the user's skin, and then the PPG sensors 105A-105D respectively detect light reflected from the different points/positions at the user's skin to measure and generate the corresponding PPG signals.

In addition, for example, the four PPG sensors 105A-105D may respectively measure and generate four PPG signals to the integration circuit 115 at a time. Each of the pressure sensors 110A-110D is located adjacent to a corresponding one of the PPG sensors 105A-105D, and is used for measuring a contact force between the user's skin and the wearable electronic device 100 when the wearable electronic device 100 is worn by the user. For example, the wearable electronic device 100 may have a square/round shape, the PPG sensor 105A and pressure sensor 110A can be neighboring to each other and located at a top position compared to the center point of the square/round shape, the PPG sensor 105B and pressure sensor 110B can be neighboring to each other and located at a right position compared to the center point, the PPG sensor 105C and pressure sensor 110C can be neighboring to each other and located at a bottom position compared to the center point, and the PPG sensor 105D and pressure sensor 110D can be neighboring to each other and located at a left position compared to the center point, as shown in FIG. 1. Alternatively, in another embodiment, the four PPG sensors 105A-105D and four pressure sensors 110A-110D may be neighboring to and located at four different corners of the center point. That is, a pressure sensor and a corresponding PPG sensor are neighboring to each other and operate/work together to respectively generate a measured contact force value and a measured waveform of the PPG signal corresponding to the measured contact force value.

The integration circuit 115 is respectively coupled to the multiple PPG sensors 105A-105D and the multiple pressure sensors 110A-110D, and it is used for receiving the measured contact forces (e.g. the measured contact force values) respectively sent from the pressure sensors 110A-110D, receiving the multiple PPG signals respectively sent from the PPG sensors 105A 105D, and for determining multiple corresponding reliability levels corresponding to the multiple PPG signals according to the multiple measured contact forces. A measured PPG signal corresponding to a high reliability level is reliable (i.e. the measured PPG signal is accurate), and another measured PPG signal corresponding to a low reliability level is unreliable (i.e. i.e. the another measured PPG signal is inaccurate).

The microcontroller 120 is coupled to the integration circuit 115, and is used for determining whether to output information of each or a PPG signal for the user according to each or a determined reliability level. The microcontroller 120 determines not outputting the information of a PPG signal for the user when a corresponding reliability level of the PPG signal is below a reference level, i.e. a low reliability level. The microcontroller 120 determines directly outputting the information of such PPG signal only when the corresponding reliability level is above the reference level, i.e. a high reliability level. By doing so, if it is determined that a measured PPG signal is not reliable, then the microcontroller 120 can determine not to output the measured PPG signal and may ask the user or inform the user of measuring the PPG signal again to try to obtain a reliable PPG signal. Only when a PPG signal is reliable, the microcontroller 120 outputs such PPG signal. Thus, the PPG signal(s) outputted from the wearable electronic device 100 for the user becomes accurate/precise result(s) since the outputted PPG signal(s) is/are reliable. That is, only reliable PPG signals can be outputted by the microcontroller 120 and displayed for the user.

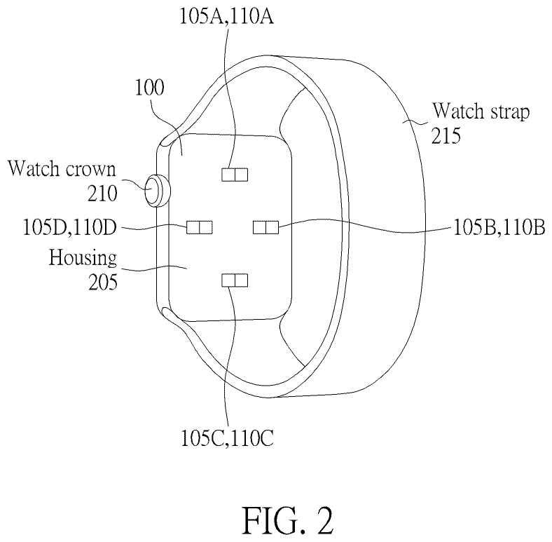

For example (but not limited), the wearable electronic device 100 may be a smart watch device which is to be worn on the user's wrist. FIG. 2 is a diagram showing an example of the wearable electronic device 100 being implemented as a smart watch device according to an embodiment of the invention. As shown in FIG. 2, the wearable electronic device 100 may be a smart watch device which comprises a housing 205, a watch crown 210, a watch strap/band 215, and the PPG sensors 105A-105D with pressure sensors 110A-110D being respectively located at different positions at the bottom side of the housing 205, as shown in FIG. 2. The PPG sensors 105A-105D with pressure sensors 110A-110D are used to respectively detect PPG signals and contact force values at corresponding different positions of the user's skin. The example is not intended to be a limitation of the invention.

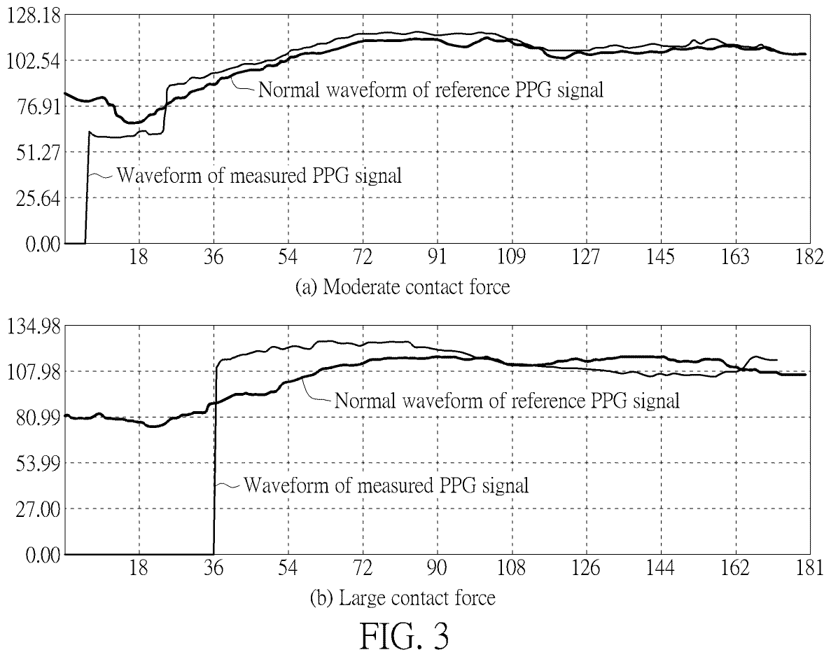

Further, in practice, when a contact force applied by the wearable electronic device 100 onto the user's skin is moderate, the PPG signal measured by a PPG sensor is accurate. If the contact force is not moderate (e. g. it is over high or over low), the measured PPG signal will be inaccurate. FIG. 3 is a diagram showing the comparison between the waveform of measured PPG signal and normal waveform of a corresponding reference PPG signal for the different examples of the moderate contact force and large contact force. As shown in the portion (a) of FIG. 3, when the contact force applied by the wearable electronic device 100 onto the user's skin is a moderate contact force, the variation between the PPG signal measured by a neighboring PPG sensor and a corresponding reference PPG signal is smaller than a reference error threshold, and thus the integration circuit 115 can determine that a reliability level of the PPG signal corresponding to the measured contact force is a high level, i.e. in this situation the generated PPG signal is accurate. Instead, as shown in the portion (b) of FIG. 3, when the contact force applied by the wearable electronic device 100 onto the user's skin is a large contact force, the variation between the PPG signal measured by a neighboring PPG sensor and another corresponding reference PPG signal is larger than the reference error threshold, and thus the integration circuit 115 can determine that a reliability level of the PPG signal corresponding to the large contact force is a low level, i.e. in this situation the generated PPG signal is inaccurate.

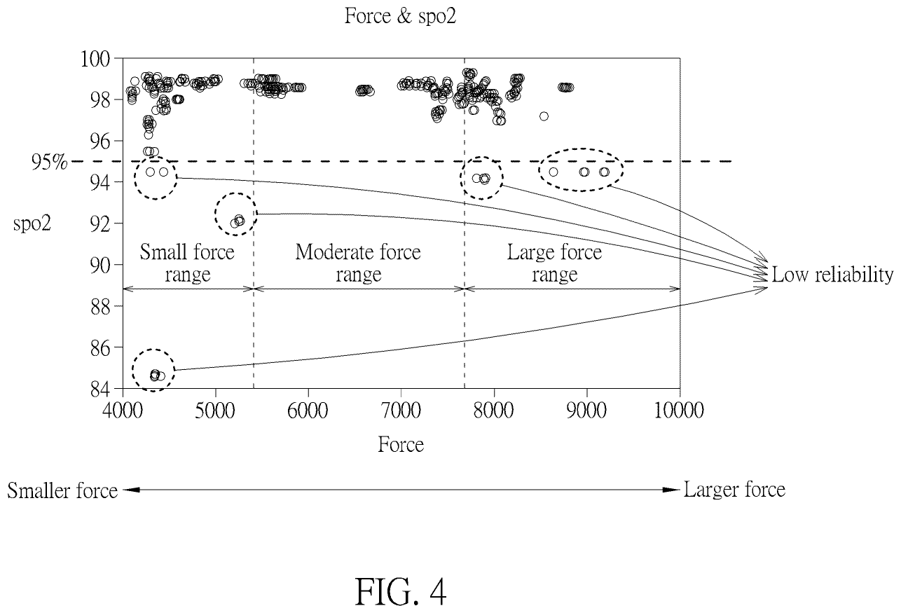

FIG. 4 is a diagram showing an example of the relation between the different oxygen saturation levels and the difference contact force values according to an embodiment of the invention. For a normal user in a normal condition, a normal person's blood oxygen level (also called as SpO₂, peripheral capillary oxygen saturation) is around 95%. As shown in FIG. 4, when the applied contact force value is within the moderate force range, the normal person's blood oxygen level can be correctly estimated to be around or above 95% based on the measured PPG signals generated from the PPG sensors 105A-105D and will not be erroneously estimated as a low level below 95%. That is, in this situation, the measured PPG signals are accurate. Instead, when the applied contact force value is within the small or large force range and not within the moderate force rage, the normal person's blood oxygen level may be erroneously estimated as a low level below 95%. That is, in this situation, the measured PPG signals may be inaccurate and unreliable.

In one embodiment, when a measured contact force is not within a moderate force range such as a default force range, the integration circuit 115 determines that a reliability level of the measured PPG signal corresponding to the measured contact force is a low level (i.e. the measured PPG signal corresponding to the measured contact force is unreliable), and then the microcontroller 120 determines not to output the information of the corresponding PPG signal for the user. In this situation, the microcontroller 120 can inform the user of adjusting a tightness of the wearable electronic device 100 and measuring the physiological feature of the user by measuring and generating the PPG signal again. For example, if the measured contact force is below the default force range, the microcontroller 120 is arranged to inform the user of tightening the wearable electronic device 100 and measuring the physiological feature of the user by measuring the PPG signal again. When the measured contact force is above the default force range, the microcontroller 120 is arranged to inform the user of loosening the wearable electronic device 100 and measuring the physiological feature of the user by measuring the at least one PPG signal again so as to obtain the accurate result of the PPG signal which is measured again.

Further, in one embodiment, the multiple pressure sensors 110A-110D are respectively located adjacent to the multiple PPG sensors 105A-105D, and the multiple pressure sensors 110A-110D are respectively used for measuring multiple contact force values and outputting the multiple contact force values into the microcontroller 120 through the integration circuit 115 which is used to generate reliability levels corresponding to the measured contact force values. The microcontroller 120 thus can sense and determine the user's operation behavior according to the measured multiple contact force values when the wearable electronic device 100 is worn by the user and then it can determine whether to inform the user of changing the user's operation behavior according to the sensed user's operation behavior.

For example, based on the measured contact forces and the corresponding reliability levels, the microcontroller 120 can determine and sense a specific kind of operation behavior of the user operating the wearable electronic device 100. For instance, when the reliability levels of the PPG signals generated from the two PPG sensors located at the two corners of the right side of the wearable electronic device 100 are high while the reliability levels of the PPG signals generated from the other two PPG sensors located at the two corners of the left side are low, the microcontroller 120 may determine that the user's behavior may tilt the wearable electronic device 100 to the left; Instead, the microcontroller 120 may determine that the user's behavior may tilt the wearable electronic device 100 to the right if the reliability levels of the PPG signals generated from the two PPG sensors located at the two corners of the right side of the wearable electronic device 100 are low while the reliability levels of the PPG signals generated from the other two PPG sensors located at the two corners of the left side are high. It should be noted, based on various combinations of the measured different contact forces and the possible reliability levels, the microcontroller 120 can correctly sense and determine the user's operation behavior.

In one embodiment, the setting of default force range can be from a cloud server through internet connection. For example, the default force range is transmitted from a host device such as a mobile device (e.g. a smart phone device) into the wearable electronic device 100 via a wireless connection between the host device and the wearable electronic device 100, and the host device obtain the default force range from the cloud server such as computer device(s) through internet connection. That is, in a preliminarily default setting, an actual value range of the default force range can be configured from the cloud server for one or more wearable electronic devices 100 respectively for one or more different users. In other words, the value range of the default force range does not need to be recorded and burn in a storage circuit within the wearable electronic device 100, and also it can be modified by the cloud server depending on the different situations.

In one embodiment, the wearable electronic device 100 can determine and set the default force range by itself. That is, after configuration, a wearable electronic device 100 may have a first default force range different from a second default force range of another wearable electronic device 100. For example, the wearable electronic device 100 comprises a training mode in which each of the multiple PPG sensors 105A 105D can measure the physiological feature for multiple times to obtain multiple PPG signals and each of the multiple corresponding pressure sensors 110A-110D can correspondingly measure the applied contact force for multiple times to obtain multiple contact force values. In this training mode, the default force range is not yet determined by the wearable electronic device 100, and the integration circuit 115 does not generate reliability levels for the PPG signals and is used to transfer the PPG signals and contact force values into the microcontroller 120 directly. The microcontroller 120 respectively calculates multiple differences (or variances) between the multiple PPG signals and a reference PPG signal (which is used as a standard and precise/accurate PPG signal), compares the differences respectively with a reference error threshold to obtain first difference values which are smaller than the reference error threshold, and determines the default force range by using first force values corresponding to the first difference values which are smaller than the reference error threshold. For example, an acceptable error (or tolerable rate) for PPG measurements may be 1%, and used as the reference error threshold. Only the first force values corresponding to the PPG signals'first difference values which are smaller than the reference error threshold such as 1% will be considered by the microcontroller 120 as moderate force values, so that the microcontroller 120 can use the moderate force values to form the default force range, i.e. a moderate force range being formed by a maximum moderate force value and a minimum moderate force value among the above-mentioned determined moderate force values. By doing this, in the normal mode of the wearable electronic device 100, the integration circuit 115 can use the determined default force range to determine reliability levels corresponding to the contact forces measured in the normal mode.

Further, in another embodiment, in the training mode, the microcontroller 120 can determine a moderate force range as the default force range based on the detected oxygen saturation levels. For example, the microcontroller 120 can respectively calculate multiple blood oxygen levels corresponding to the multiple PPG signals based on the PPG signals, compare the multiple blood oxygen levels respectively with a reference oxygen saturation level to obtain abnormal blood oxygen levels which are smaller than the reference oxygen saturation level so as to obtain an abnormal blood oxygen range which covers the abnormal blood oxygen levels. For example, a normal oxygen saturation level, i.e. a standard blood oxygen level for a generally healthy person, is at a normal range between 95% and 100%. The reference oxygen saturation level may be configured as 95%, i.e. the minimum value of the normal range between 95% and 100%, and is used as a minimum threshold of a normal blood oxygen level. The microcontroller 120 can compare each of the calculated blood oxygen levels with the reference oxygen saturation level such as 95% to determine and obtain the abnormal blood oxygen levels which are smaller than 95%, so that the abnormal blood oxygen range, which covers the abnormal blood oxygen levels, can be formed and obtained. For instance, the maximum value of the calculated abnormal blood oxygen levels may be 94% for a user, and thus the abnormal blood oxygen range is determined to be between 0% and 94% for the user. In a different situation, the maximum value of the calculated abnormal blood oxygen levels may be very approximate to 95% for another user, and thus the abnormal blood oxygen range is determined to be between 0% and 95% for the another user. p After obtaining the abnormal blood oxygen range, the microcontroller 120 can determine a normal blood oxygen range by excluding the abnormal blood oxygen range and then determine the default force range by using a force range corresponding to the determined normal blood oxygen range. For example, the abnormal blood oxygen range is determined to be between 0% and 94% for the user, and thus the normal blood oxygen range by excluding the abnormal blood oxygen range is between 94% and 100%. In another example, the abnormal blood oxygen range is determined to be between 0% and 95% for the another user, and thus the normal blood oxygen range by excluding the abnormal blood oxygen range is between 95% and 100%. Finally, after determining the normal blood oxygen range, the microcontroller 120 can obtain all possible force values corresponding to the all blood oxygen values in the determined normal blood oxygen range, determine which force value range can cover the all possible force values, and then can use the determined force value range as the default force range (i.e. the calculated moderate force range).

In addition, in one embodiment, the above-mentioned reference oxygen saturation level may be finely adjusted in response to a different altitude condition. For example, in a high-altitude condition, a normal oxygen saturation level may range from 90% to 95%, and thus the reference oxygen saturation level may be adjusted down to for example 90% (but not limited) in this situation. Similarly, the above-mentioned normal blood oxygen range can be finely adjusted.

Further, in another embodiment, the default force range may be identical for different users and then can be adjusted by the microcontroller 120 as different ranges for the different users. For example, the microcontroller 120 is used to adjust the default force range as a first force range and a second force range respectively for a first user and a second user, and the first force range and the second force range are different.

Further, in one embodiment, the microcontroller 120 in the training mode can determine the default force range simultaneously based on the detected oxygen saturation levels and the calculated differences (or variances) between the multiple PPG signals and the reference PPG signal. The operations are similar to those mentioned in the above paragraphs and are not detailed for brevity.

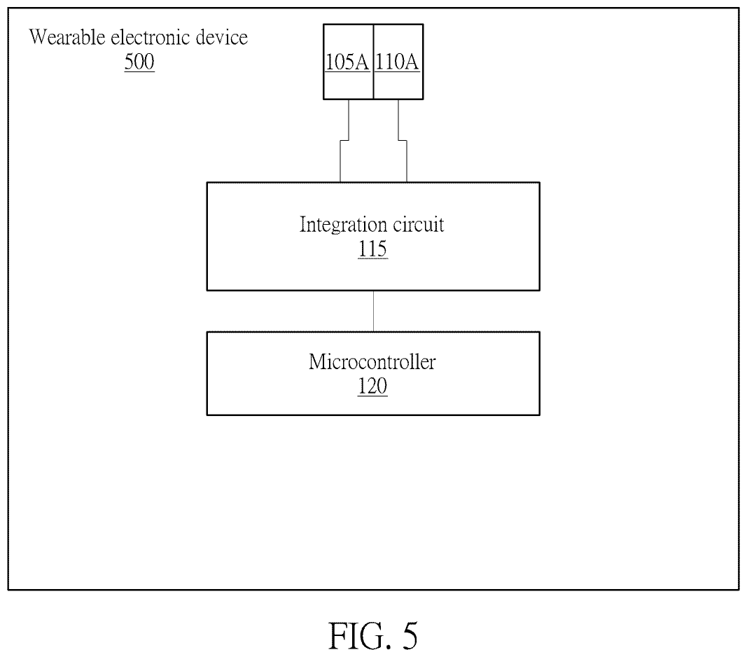

Additionally, in one embodiment, the number of pressure sensor may be configured as one. FIG. 5 is a block diagram of a wearable electronic device 500 according to another embodiment of the invention. As shown in FIG. 5, the wearable electronic device 500 may be a smart ring device (but not limited) and comprises only one PPG sensor 105A, only one corresponding pressure sensor 110A, the integration circuit 115, and the microcontroller 120. This modification also falls within the scope of the invention.

Those skilled in the art will readily observe that numerous modifications and alterations of the device and method may be made while retaining the teachings of the invention. Accordingly, the above disclosure should be construed as limited only by the metes and bounds of the appended claims.

1. A wearable electronic device, comprising:
at least one photoplethysmogram (PPG) sensor, for sensing a physiological feature of a user by measuring at least one PPG signal by illuminating a user's skin and measuring changes in light absorption based on at least one light reflected from the user's skin when the wearable electronic device is worn by the user;
at least one pressure sensor, located adjacent to the at least one PPG sensor with no other sensor disposed therebetween, for measuring at least one contact force between the user's skin and the wearable electronic device when the wearable electronic device is worn by the user;
an integration circuit, coupled to the at least one PPG sensor and the at least one pressure sensor, for determining at least one reliability level corresponding to the at least one PPG signal according to the at least one measured contact force; and
a microcontroller, coupled to the integration circuit, for determining whether to output information of the at least one PPG signal for the user according to the at least one determined reliability level.

2. The wearable electronic device of claim 1, wherein when the at least one measured contact force is not within a default force range, the at least one determined reliability level is a low level, and the microcontroller determines not to output the information of the at least one PPG signal for the user; and, the default force range is a moderate force range.

3. The wearable electronic device of claim 2, wherein the microcontroller is arranged to inform the user of adjusting a tightness of the wearable electronic device and measuring the physiological feature of the user by measuring the at least one PPG signal again.

4. The wearable electronic device of claim 3, wherein when the at least one measured contact force is below the default force range, the microcontroller is arranged to inform the user of tightening the wearable electronic device and measuring the physiological feature of the user by measuring the at least one PPG signal again; and, when the at least one measured contact force is above the default force range, the microcontroller is arranged to inform the user of loosening the wearable electronic device and measuring the physiological feature of the user by measuring the at least one PPG signal again.

5. The wearable electronic device of claim 2, wherein the default force range is transmitted from a host device into the wearable electronic device via a wireless connection between the host device and the wearable electronic device, and the host device obtain the default force range from a cloud server through internet connection.

6. The wearable electronic device of claim 2, wherein the wearable electronic device comprises a training mode in which the at least one PPG sensor measures the at least one PPG signal for multiple times to obtain multiple PPG signals and the at least one pressure sensor correspondingly measures the at least one contact force for multiple times to obtain multiple contact forces, and the microcontroller respectively calculates multiple differences between the multiple PPG signals and a reference PPG signal, compares the difference with a reference error threshold to obtain first difference values which are smaller than the reference error threshold, and determines the default force range by using first force values corresponding to the first difference values which are smaller than the reference error threshold.

7. The wearable electronic device of claim 2, wherein the wearable electronic device comprises a training mode in which the at least one PPG sensor measures the at least one PPG signal for multiple times to obtain multiple PPG signals and the at least one pressure sensor correspondingly measures the at least one contact force for multiple times to obtain multiple contact forces, and the microcontroller respectively calculates multiple blood oxygen levels corresponding to the multiple PPG signals, compares the multiple blood oxygen levels with a reference oxygen saturation level to obtain abnormal blood oxygen levels which are smaller than the reference oxygen saturation level so as to obtain an abnormal blood oxygen range which covers the abnormal blood oxygen levels, and determines a normal blood oxygen range by excluding the abnormal blood oxygen range and then determines the default force range by using a force range corresponding to the determined normal blood oxygen range.

8. The wearable electronic device of claim 2, wherein the microcontroller is used to adjust the default force range as a first force range and a second force range respectively for a first user and a second user, and the first force range and the second force range are different.

9. The wearable electronic device of claim 1, wherein the wearable electronic device comprises multiple PPG sensors and multiple pressure sensors respectively located adjacent to the multiple PPG sensors; the multiple pressure sensors are respectively used for measuring multiple contact forces and outputting the multiple contact forces into the microcontroller, and the microcontroller is used to sense the user's operation behavior when the wearable electronic device is worn by the user according to the measured multiple contact forces and then determines whether to inform the user of changing the user's operation behavior according to the sensed user's operation behavior.

10. The wearable electronic device of claim 1, to be worn on the user's wrist.

11. A method of a wearable electronic device, comprising:
providing at least one photoplethysmogram (PPG) sensor to sense a physiological feature of a user by measuring at least one PPG signal by illuminating a user's skin and measuring changes in light absorption based on at least one light reflected from the user's skin when the wearable electronic device is worn by the user;
using at least one pressure sensor, located adjacent to the at least one PPG sensor with no other sensor disposed therebetween, to measure at least one contact force between the user's skin and the wearable electronic device when the wearable electronic device is worn by the user;
determining at least one reliability level corresponding to the at least one PPG signal according to the at least one measured contact force; and
determining whether to output information of the at least one PPG signal for the user according to the at least one determined reliability level.

12. The method of claim 11, further comprising:
determining that the at least one determined reliability level is a low level and not to output the information of the at least one PPG signal for the user when the at least one measured contact force is not within a default force range, the default force range being a moderate force range.

13. The method of claim 12, further comprising:
informing the user of adjusting a tightness of the wearable electronic device and measuring the physiological feature of the user by measuring the at least one PPG signal again.

14. The method of claim 13, further comprising:
when the at least one measured contact force is below the default force range, informing the user of tightening the wearable electronic device and measuring the physiological feature of the user by measuring the at least one PPG signal again; and
when the at least one measured contact force is above the default force range, informing the user of loosening the wearable electronic device and measuring the physiological feature of the user by measuring the at least one PPG signal again.

15. The method of claim 12, wherein the default force range is transmitted from a host device into the wearable electronic device via a wireless connection between the host device and the wearable electronic device, and the host device obtain the default force range from a cloud server through internet connection.

16. The method of claim 12, wherein the wearable electronic device comprises a training mode in which the at least one PPG sensor measures the at least one PPG signal for multiple times to obtain multiple PPG signals and the at least one pressure sensor correspondingly measures the at least one contact force for multiple times to obtain multiple contact forces, and the method further comprises:
respectively calculating multiple differences between the multiple PPG signals and a reference PPG signal;
comparing the difference with a reference error threshold to obtain first difference values which are smaller than the reference error threshold; and
determining the default force range by using first force values corresponding to the first difference values which are smaller than the reference error threshold.

17. The method of claim 12, wherein the wearable electronic device comprises a training mode in which the at least one PPG sensor measures the at least one PPG signal for multiple times to obtain multiple PPG signals and the at least one pressure sensor correspondingly measures the at least one contact force for multiple times to obtain multiple contact forces, and method further comprises:
respectively calculating multiple blood oxygen levels corresponding to the multiple PPG signals;
comparing the multiple blood oxygen levels with a reference oxygen saturation level to obtain abnormal blood oxygen levels which are smaller than the reference oxygen saturation level so as to obtain an abnormal blood oxygen range which covers the abnormal blood oxygen levels; and
determining a normal blood oxygen range by excluding the abnormal blood oxygen range and then determines the default force range by using a force range corresponding to the determined normal blood oxygen range.

18. The method of claim 12, further comprising:
adjusting the default force range as a first force range and a second force range respectively for a first user and a second user, the first force range and the second force range being different.

19. The method of claim 11, wherein the wearable electronic device comprises multiple PPG sensors and multiple pressure sensors respectively located adjacent to the multiple PPG sensors, and the method further comprises:
using the multiple pressure sensors to measure multiple contact forces and outputting the multiple contact forces into the microcontroller; and
sensing the user's operation behavior when the wearable electronic device is worn by the user according to the measured multiple contact forces and then determining whether to inform the user of changing the user's operation behavior according to the sensed user's operation behavior.

20. The method of claim 11, to be worn on the user's wrist.
